SEQUENCE LISTING (1 sequence)

SEQ ID NO: 1 - DNA, 401 bp, Homo sapiens
aggggtagaagaacttgactttggggagactgagaaagaccacatgtaagagagagagacatggaaaatgaggtagaaacagacagaaacaaagaaaggcacaaaaattaagaggcagaggaaaggtgaaaatagccaggagacagaaactgcaggatgagagaaatgaatagagagagacagagagactaagacagcaaaaggcaggaaggaaaaacagaaaatgagaaggtgtgagagagagagagatggagaggtggagaacagagagactgaaatgagaaagagacctctcggagagagaccgagaggaagaggctggcagagggagagggtcagagaccaaagacagagacaaaaagagaaatagggttttgcaaagactgactgacagaaacaga

(19) United States
(12) Patent Application Publication
(10) Pub. No.: US 2010/0136532 A1
(43) Pub. Date: Jun. 3, 2010

(54) AQP5 Polymorphism

(75) Inventors: Michael Adamzik, Essen (DE); Ulrich Frey, Essen (DE); Winfried Siffert, Gelsenkirchen (DE)

Correspondence Address: AMSTER, ROTHSTEIN & EBENSTEIN LLP, 90 PARK AVENUE, NEW YORK, NY, 10016, US

(73) Assignee: UNIVERSITÄT DUISBURG-ESSEN, Essen (DE)

(21) Appl. No.: 12/308,613

(22) PCT Filed: Jun. 12, 2007
(86) PCT No.: PCT/EP2007/055731

§ 371 Date: Oct. 26, 2009

(30) Foreign Application Priority Data
Jun. 19, 2006 (DE) 10 2006 028 009.1

(51) Int. Cl. C12Q 1/68

(52) U.S. Cl. 435/6

(57) ABSTRACT

In order to predict disease risks, disease courses and the response of an individual patient to pharmacological and non-pharmacological therapies a polymorphism in the AQP5 gene has been investigated, in particular in the promoter region of said gene,on the human 12q13 chromosome, wherein the therapy can also be a cosmetic treatment.

The invention relates to the use of a genetic alteration in the human AQP5 gene for predicting disease risks and disease courses and for predicting a patient's response to treatments or measures for influencing the quality or appearance of human skin.

Aquaporins (AQP) form a family of at least ten integral membrane proteins which transport water and in some cases also anions or low-molecular substances such as glycerol (1). In mammals, they are expressed in epithelial, endothelial and other tissues in which the transport of water plays a role, but also in skin tissue and fatty tissue. Functional studies suggest that aquaporins 1, 2, 4, 5 and 8 are primarily water-selective, whereas aquaporins 3, 7, 9 and 10 (“aquaglyceroporins”) also transport glycerol and other small molecules. At least six AQPs including AQP1-4, 6 and 7, are expressed in the kidneys.

Aquaporins in all tissues are of physiological importance, but aquaporins are especially important in tissues where there is a strong physiological water flow. In humans, aquaporins regulate the water balance of erythrocytes and cells in the kidneys (2), eyes (3), brain (4) and inner ear cochlea (5). In hepatic biliary ducts and the gallbladder, aquaporins are responsible for the concentration and secretion of bile fluid. In the central nervous system, cells that secrete cerebrospinal fluid contain water channels that play an important role in the function of the blood-brain barrier. In the cells of blood capillaries, they regulate the influx and outflow of the intercellular fluid. In the pulmonary vesicles, they ensure the fluid film required for gas exchange.

Functional classification of aquaporins In the past has been based on two groups, namely (a) pure aquaporins (AQP-1, 2, 4, 5, 6), and (b) aquaporins that also allow small uncharged molecules such as glycerol and urea to pass through in addition to water (AQP-3, 7, 9, 10). A new dimension of the aquaporin function was recently revealed through the discovery that NO and CO₂ gas pass through AQP-1 (6) and that AQP-6 conducts anions (7). Aquaporins in general influence the water and electrolyte balance and also influence anion transport and glycerol transport.

Polymorphism of the AQP5 gene is already present in some SNP databases, although it has not been assigned a function, nor has any molecular or diagnostic significance been attributed to it.

The object of the present invention is to provide a diagnostic means for predicting disease risks and disease courses and for predicting a patient's response to treatments or measures for influencing the quality or appearance of human skin.

This object is achieved by detection of a polymorphism in the AQP5 gene on human chromosome 12q13 for prediction of disease risks and disease courses and the response of an individual patient to pharmacological and nonpharmacological treatments. This object is also achieved by detection of a polymorphism in the promoter region of the AQP gene for the aforementioned purposes, preferably the A(−1364)C polymorphism being detected.

AQP5 occurs in the apical membranes of the terminations of glands such as the salivary glands, lachrymal glands, sweat glands and other glands. This is the last membrane through which water passes during production of saliva, tears and other secretions. Furthermore, AQP5 has also been detected in the inner ear, brain, kidney, stomach, eye, ovaries, colon, uterus, bladder, skin and lungs. Ubiquitous expression of AQP5 may be assumed. Absence of AQP5 and its effects on the body have recently been investigated in so-called knockout models on mice or rats, in which absence of AQP5 was found to lead to diseases of the lungs, eyes, teeth and glands. In the absence of AQP5, disturbed pulmonary water regulation and hyperreactivity of the respiratory tract (asthma, hayfever) to chemical stimuli in the lungs were found (8, 9). In the eye, there were changes in the cornea, and animals without AQP5 had an increased risk of caries.

Investigations on human tissues have shown that tumorigenesis (e.g., carcinoma of the colon and ovaries) is associated with an altered AQP5 gene expression which promotes tumor growth in the early stages (10). Expression of AQP5 can be regulated by substances which have a direct or indirect influence on the intracellular cAMP level (8) or cGMP level (11) or on the activity of protein kinases. Such substances include beta-adrenergic agonists (dobutamine, isoprenaline, terbutaline), beta-blockers (propranolol) as well as vasopressin and prostaglandins.

The present invention thus relates in particular to:

a) providing function-altering genomic polymorphisms and haplotypes in the AQP5 gene, which lead either to an amino acid exchange, or

b) influencing the splicing behavior, or

c) leading to a change in protein expression or to a change in the expression of spliced variants, or

d) being suitable for discovering and/or validating other polymorphisms and/or haplotypes in the AQP5 gene;

e) providing nucleotide exchanges and haplotypes suitable for predicting disease risks and prognoses in general,

f) providing nucleotide exchanges and haplotypes suitable for predicting in general the response to pharmaceutical drugs and the adverse effects thereof,

g) providing nucleotide exchanges and haplotypes suitable in general for predicting the effect of other forms of treatment (e.g., irradiation, heat, cold, motion).

Because of the fundamental importance of AQP5 for signal transduction, the water and electrolyte balance and the fat metabolism, such polymorphisms or haplotypes have proven suitable for predicting disease risks and disease courses in all diseases or predicting therapeutic responses/failures or adverse effects for all pharmaceutical drugs or nonpharmacological treatments. In addition, it is possible to identify people who will respond to inhibition of APQ5 in a particular manner. Furthermore, the water content also determines the degree of swelling of cells, including the skin. It is thus possible to identify people in whom fundamental properties of the skin (wrinkles, thickness of subcutaneous fatty tissue, degree of hydration) are altered and who respond in a particular manner to topical application of dermatologic preparations, which also includes skin care cosmetics, creams and lotions.

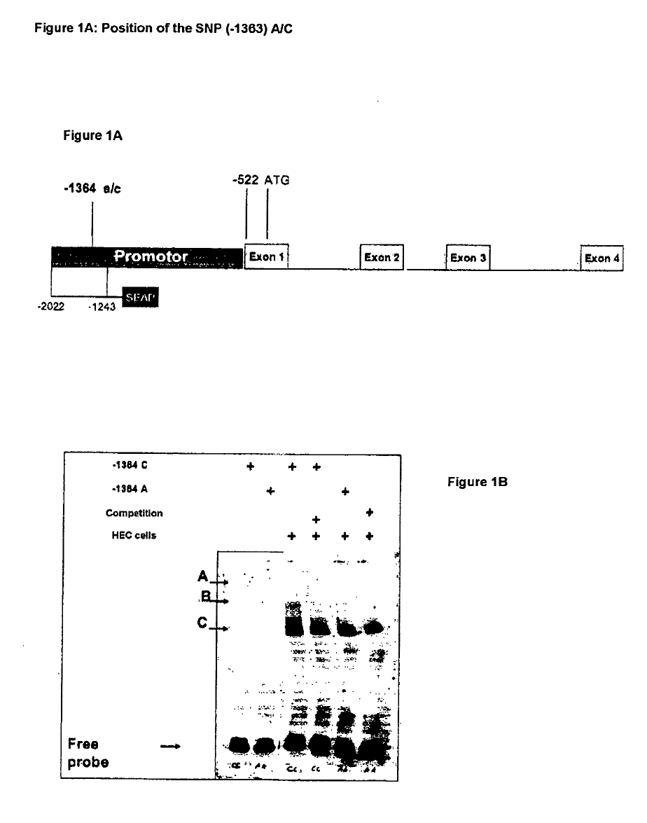

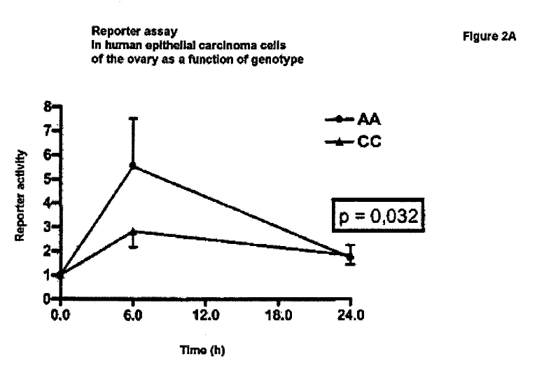

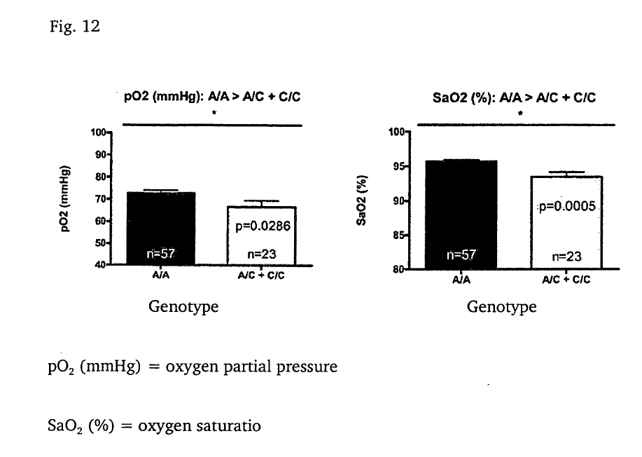

The human gene APQ5 is localized on chromosome 12q13 (accession no.: NM_{—}001651 of the gene bank of the National Center for Biotechnology Information (NCBI)). By systematic sequencing of DNA samples of humans (FIG. 1A) and database analysis, the A(−1364)C gene polymorphism (dbSNP (SNP database of the NCBI): rs 3759129 has been localized in front of exon-1 in the promoter of the gene. Meanwhile, a substitution of adenine by cytosine occurs in the promoter region at position −1364. FIG. 8 illustrates a detail from chromosome 12, comprising nucleotides 48640504-486408904.

To determine the polymorphism, gene sequences that occur before exon-1 of AQP5 have been amplified by PCR reaction and sequenced by the method according to Sanger. Those skilled in the art are familiar with the methods required for this, such as derivation of the primer pairs required for PCR reaction and selection of sequencing primers.

Alternative numbering for this SNP is provided by assigning the number +1 to nucleotide A of start codon ATG. By convention, there is no number 0, so the number −1 is assigned to the nucleotide situated in front of the A of the start codon. This would thus provide alternative nomenclature for the A(−842)C polymorphism.

Detection of these SNPs in the sense of an inventive use can be performed with any methods with which those skilled in the art are familiar, e.g., direct sequencing, PCR with a subsequent restriction analysis, reverse hybridization, dot-blot or slot-blot methods, mass spectrometry, Tagman® or Light-Cycler® technologies, Pyrosequencing®, Invader® technology and Luminex methods. Furthermore, such genetic polymorphisms may be detected at the same time by multiplex PCR and hybridization on a DNA chip.

### Distribution of the A(−1364)C Polymorphism in Different Ethnicities and Use of these Genotypes to Discover Other Relevant Polymorphisms and Haplotypes

To do so, genotyping was performed on various DNA samples from Caucasians and Black Africans and Chinese volunteers. The results are summarized in the following Table 1.

TABLE 1

| | White Caucasians | Black Africans |
|---|---|---|
| N(%) | 94 | 72 |
| CC | 2 (3.9%) | 0 |
| AC | 33 (31.6%) | 7 (9.7%) |
| AA | 59 (64.5%) | 65 (90.3%) |
| % C | 19.7 | 4.9 |

This genotype distribution is different and highly significant in the χ² test, with χ=16.6 and P=0.0003. The A allele occurs the most commonly among Black Africans. One may conclude from this distribution that the AA genotype is the “original state” in terms of developmental history, based on Caucasians. Such differences in genotype distribution in different ethnicities usually indicate that associated phenotypes had significance for evolution and brought a certain advantage to the carriers. Those skilled in the art are aware that ethnically different genotype distributions are an indication that even today certain genotypes and haplotypes are associated with certain diseases or physiological and pathophysiological responses or responses to treatment, e.g., with pharmaceutical drugs.

It is a subject matter of the present invention that this polymorphism can be utilized to detect and validate other relevant genomic gene alterations in AQP5 or neighboring genes which are in a coupling equilibrium with genotypes in the AQP5 gene, for example. These may also be genes located on chromosome 12, but at a great distance from the AQP5 gene. The following procedure is observed in this regard.

First, for certain phenotypes (cellular properties, disease states, disease courses, drug responses) an association with A(−1364)C polymorphism is established.

Then for the newly detected gene alterations in AQP5 or neighboring genes, there is an investigation of whether existing associations are weakened or strengthened by using the genotypes or haplotypes described above.

### Functional Importance of The A(−1364)C Polymorphism

The question of which functional gene alterations in the AQP5 gene are to be assigned was investigated. For example, a correlation with alternative splicing, tissue-specific expression or overexpression of the AQP5 protein as a function of genotypes of A(−1364)C polymorphism was to be investigated here. First, a computer program was used to investigate whether the nucleotide exchanges that had been found could influence the binding of transcription factors. Transcription factors bind to specific consensus sequences and can increase or reduce the promoter activity, resulting in either increased or decreased transcription of the gene and thus increasing or decreasing the expression level of the coded protein.

For experimental investigation of this effect, a so-called EMSA (electrophoretic mobility shift assay) is performed. Those skilled in the art are familiar with the relevant method. In this experiment, short nucleic acid sections containing the polymorphism were incubated with cell nucleus extracts, namely in this case from HEC cells. Transcription factor proteins in these extracts then bind to nucleic acid sections with differing intensities. Binding to the DNA is then visualized in X-ray film. An intense band results here from a strong binding. FIG. 1B shows the result of this experiment with specific constructs containing either the AA genotype or the CC genotype. The stronger intensity of the CC construct band (band B in FIG. 1B, lane 3) proves stronger binding of a transcription factor in this region in the CC genotype in comparison with the AA genotype (FIG. 1B, band B, volume 5). The disappearance of the band due to a complementary oligonucleotide (FIG. 1B, lanes 4 and 6, band B) proves the specificity of the bond.

For functional detection of a modified promoter activity as a function of certain genotypes, different fragments of the promoter (nt-2002-nt-1243; FIG. 1A) were cloned with the CC or AA genotype in the vector pSEAP to quantify the promoter activity by means of a so-called “reporter assay” after expression of the vector in ovarian carcinoma cells (FIG. 2A). To do so, the constructs were cloned in front of a gene that codes for secreted alkaline phosphatase (SEAP). If the construct has a promoter activity, the SEAP gene is transcribed to an increased extent and the increased secretion of alkaline phosphatase into the cellular culture medium is measurable. As FIG. 2A shows, the construct with the AA genotype has a definitely increased reporter activity in comparison with the CC construct. The A(−1364)C polymorphism in the promoter of the AQP5 gene thus leads to the promoter activity being increased with the AA genotype and thus the AQP5 protein being expressed to an increased extent. To check on whether this regulation also takes place in vivo, expression of AQP5 was also investigated on an mRNA level by means of real-time PCR in cardiac tissue.

To do so, mRNA from human surgical tissue was obtained from heart surgeries and transcribed in cDNA by means of reverse transcriptase. Those skilled in the art are familiar with this method. The expression level was then determined by real-time PCR (Tagman method) and compared with the expression level of the β-actin housekeeping gene. The results are shown in FIG. 2B. The AA genotype leads to an increase of at least 50% in the AQP5 transcription in comparison with the C allele. In this case, the less common homozygous CC genotypes were analyzed in combination with the heterozygous AC genotypes.

### Importance of A(−1364)C Polymorphism for the Cardiovascular System

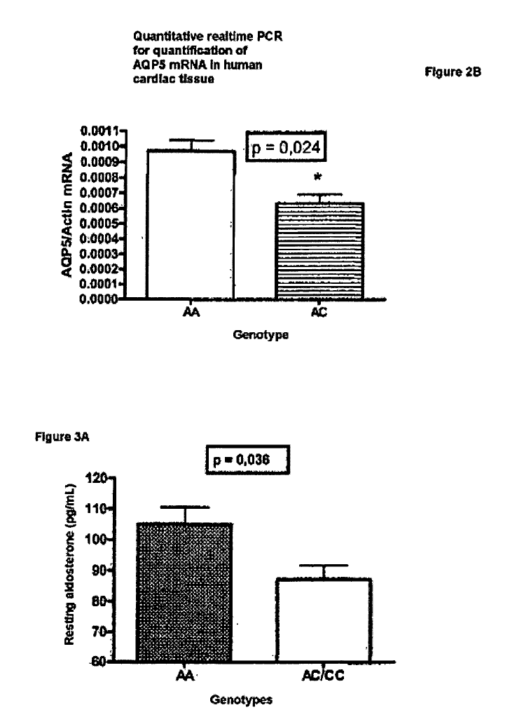

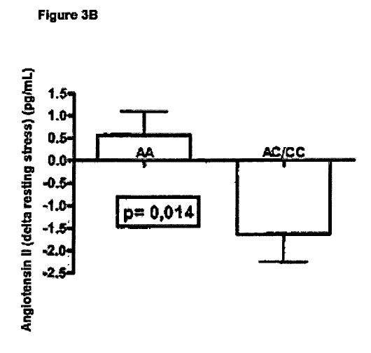

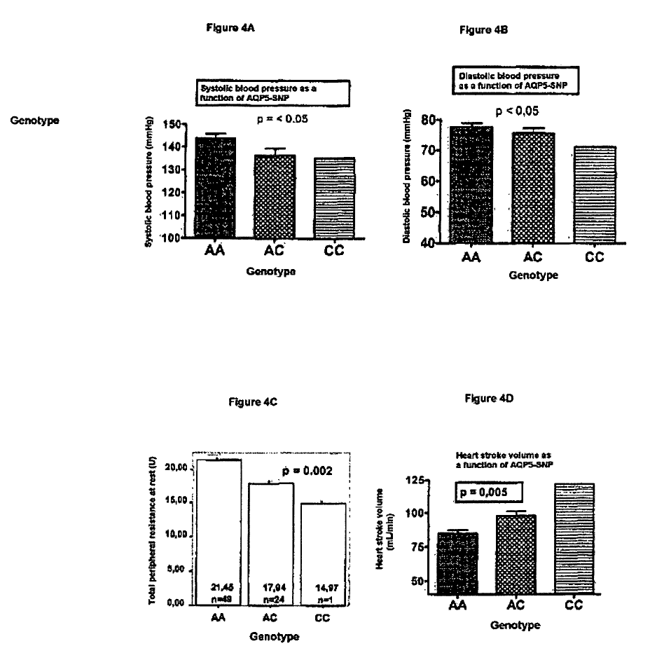

Blood pressure regulation is closely related to the sodium and water balance. This is in turn regulated by various hormone systems, including the sympathetic nervous system, the renin-angiotensin-aldosterone system and the ADH-vasopressin system. Changes in expression of aquaporins must therefore also influence these systems. As shown in FIG. 3a, significantly elevated aldosterone levels are found in humans having the AA genotype in comparison with those with the AC/CC genotype. Under dobutamine loading, stronger suppression of the angiotensin II hormone is definitely found in those with the AC/CC genotype than those with the AA genotype (FIG. 3B). In agreement with these findings, A(−1364)C polymorphism is associated with significant changes in the diastolic and systolic blood pressure and the total peripheral resistance, such that persons having the AA genotype have the highest blood pressure levels with a statistical significance. Thus the systolic values even in healthy young volunteers amount to 143.5±14.9 mmHg (AA genotype), 136.9±15.8 mmHg (AC genotype) and 153 mmHg (CC genotype; FIG. 4a). The AA genotype can thus already be classified as hypertensive. The diastolic levels are also significantly different with average values of 77.6±9.4 mmHg (AA genotype), 75.6±8.8 mmHg (AC genotype) and 71 mmHg (CC genotype; FIG. 4b). In agreement with these findings, the following holds for the total peripheral resistance: AA>AC>CC (FIG. 4c). Furthermore, the heart stroke volume statistically significantly depends on the AQP5 genotype (FIG. 4d). This amounts to 84.6±19.7 mL (AA genotype), 97.86±17.5 mmHg (AC genotype) and 122 mmHg (CC genotype; p=0.005).

It is possible to deduce from these findings that people with the (−1364) AA/AC genotypes have a modified sodium and water balance based on altered AQP5 expression and therefore have altered concentrations of cardiovascular hormones such as aldosterone and angiotensin II. This means an overall increase in cardiovascular risk. As shown here (FIG. 4), there is a risk of hypertension and a reduced heart stroke volume. Hypertension is in turn one of the main risk factors for strokes and transient ischemic attacks as well as cerebral hemorrhage, myocardial insufficiency with or without pulmonary edema, coronary heart disease, myocardial infarction, left heart hypertrophy, arrhythmias (e.g., atrial fibrillations, ventricular tachycardia), renal damage, eye damage (e.g., hypertensive retinopathy), Alzheimer's disease and other forms of dementia (e.g., microvascular encephalopathy), loss of hearing, general atherosclerosis, aneurysms of the major arteries, gestosis and pre-eclampsia. To verify this hypothesis, genotyping was performed on a population of 93 patents with coronary heart disease and the genotype distribution was compared with that of the healthy control population. The results are shown in Table 2.

TABLE 2

| | Healthy controls | CHD patients |
|---|---|---|
| n | 94 | 93 |
| CC | 2 (2.1%) | 5 (5.4%) |
| AC | 33 (35.1%) | 17 (18.3%) |
| AA | 59 (62.8%) | 71 (76.9%) |
| % C | 19.7 | 14.5 |

The genotype distribution in Table 2 shows an increased incidence of AA genotypes in CHD patients, and the genotype distribution is significantly different (p=0.023; χ²=7.5, 2 df). AA genotypes thus have an increased risk of coronary heart disease in comparison with healthy controls, which can be expressed as an odds ratio (OR) as follows: OR AA/AC=2.3 (95% CI=1.2-4.6; p=0.013) and OR AA versus AC plus CC=1.9 (95% CI=1.01-3.61; p=0.044).

Within the group of these CHD patients, a subcohort had already suffered one myocardial infarction.

TABLE 3

| | CHD patients with | CHD patients with a |
|---|---|---|
| | no infarction | myocardial infarction |
| n | 94 | 72 |
| CC | 3 (6.4%) | 2 (4.3%) |
| AC | 13 (27.7%) | 4 (8.7%) |
| AA | 31 (65.9%) | 40 (87%) |
| % C | 19.7 | 4.9 |

Table 3 shows another high incidence of the AA genotype in patients with a history of myocardial infarction. Thus AA genotypes have an increased risk of myocardial infarction which can be expressed as an odds ratio (OR) as follows: OR AA versus AC plus CC=3.4 (95% CI=1.2-9.8; p=0.017).

On the whole, this proves that genotyping of the A(−1364)C polymorphism is suitable for predicting the risk of occurrence of the aforementioned diseases but also their progression. This has been demonstrated here on the example of the myocardial infarction which is a result of coronary heart disease.

### The Meaning of A(−1364)C Polymorphism for Treatment of Cardiovascular Diseases

The present invention also relates to the use of detection of the genotype of the A(−1364)C polymorphism for a targeted pharmacological or nonpharmacological treatment of the diseases of the cardiovascular system described above. In the past, such treatment has been based on the guidelines of professional medical organizations without taking into account specific deviations in the sodium and water balance, e.g., deviations also caused by genetic factors. This is where the detection of specific genotypes of A(−1364)C polymorphism can contribute toward predicting the effect of pharmaceutical drugs and other measures which intervene in the regulation of the water balance, the electrolyte balance, blood pressure, force of myocardial contraction, heart rate, organ circulation and blood volume. Furthermore, optimal doses, duration of treatment and possible adverse effects can be predicted in this way. These pharmaceutical drugs include in particular:

diuretics, e.g., loop diuretics, thiazide and potassium-sparing diuretics (influencing the electrolyte and water balance);

aldosterone antagonists, e.g., spironolactone, eplerenone (influencing the electrolyte and water balance);

ACE inhibitors, e.g., captopril, enalapril (influencing the formation of angiotensin and aldosterone);

renin antagonists; inhibiting the effect of the RAS;

angiotensin receptor blockers, e.g., losartan and other sartans;

endothelin agonists and antagonists and endothelin receptor blockers;

calcium channel blockers (antihypertensive, leading to a reactive activation of the RAS, increase in volume);

alpha-adrenoceptor blockers (antihypertensive, leading to a reactive activation of the RAS; increase in volume);

beta-adrenoceptor blockers (antihypertensive, inhibiting the release of renin);

potassium channel openers, e.g., moxonidine (antihypertensive, leading to a reactive activation of the RAS, increase in volume);

CNA-active sympathomimetics, e.g., clonidine;

dihydralazine;

nitrates (e.g., vasodilating, increase in the cGMP concentration);

phosphodiesterase inhibitors, in particular those that inhibit cGMP and cAMP phosphodiesterases (e.g., sildenafil, vardenafil).

### Importance of the A(−1364)C Polymorphism for Regulation of Body Weight and Skin Properties

Aquaporins can transport not only water but also glycerol and other low-molecular substances. Aquaporins thus contribute toward regulation of body weight, while on the other hand also contributing toward the elasticity and glycerol content of skin. However, this has been demonstrated so far only for aquaporins 3 and 7 (12-17). In this case, there was an investigation of how the AQP5 polymorphism described here has an influence on the body mass index (BMI), which is a measure of body fat content, and the thoracic skin fold thickness, which is a measure of fat content and hydration state. As shown in FIG. 5a, the BMI of young healthy volunteers with the AA genotype is much higher (BMI=24.3±2.7 kg/m²) than in volunteers who have the AC/CC genotypes (23.1±2.3 kg/m²). Furthermore, the skin fold thickness is much greater (2.11±0.9 cm) in volunteers with the AA genotype than in those with the AC/CC genotypes (1.7±0.8 cm; FIG. 5b). It can be deduced from this that AA genotypes have a significantly increased risk of obesity, which in turn leads to an increased risk of the sequelae associated with obesity including diabetes, gout, arterial damage, hypertension and cancer. This risk can also be predicted by genotyping of the A(−1364)C polymorphism. In addition, genotyping allows an improved prediction of the causes of obesity and diabetes and the use of specific therapeutic measures, e.g., medication or physical activity. Furthermore, use of the present invention as intended allows predictions about the hydration state and elasticity of skin and the development of specific measures of a medicinal or cosmetic type for optimization of the composition of skin. These also include, for example, developing specific cosmetics for tightening skin, increasing its water and fat content, which can then be used in a targeted manner in those with specific genotypic characteristics.

### Importance of the A(−1364)C Polymorphism for Cancer Diseases

Some aquaporins are overexpressed in carcinoma tissue (18; 19) (10; 20) and aquaporins contribute toward tumor progression by influencing angiogenesis, for example (21). Therefore aquaporins play an important role in the development and progression of tumors, they limit the survival of tumor patients and they determine the response to medicinal and nonmedicinal cancer treatments.

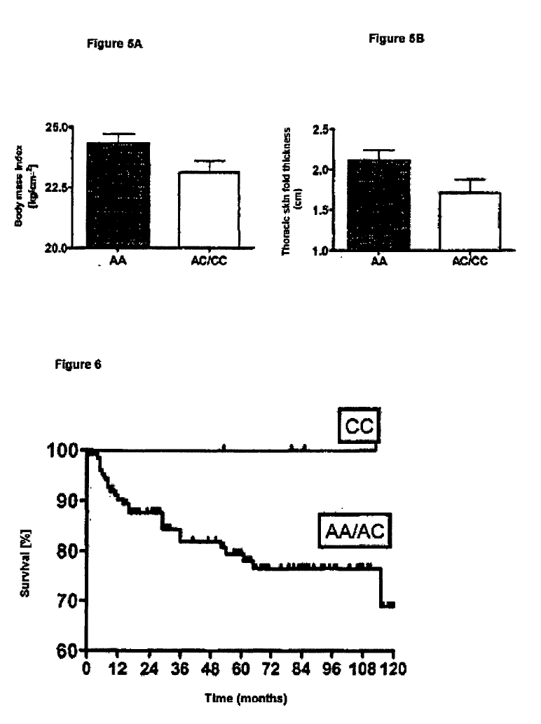

Patients with renal cell carcinoma were genotyped and the genotypes were correlated with their survival. As shown in FIG. 6, patients with the CC genotype did not die within the observation period of 10 years, whereas only approximately 70% of the patients with the AA/AC genotypes survived for the same period of time. This shows that overexpression of AQP5 in humans correlates with an increased tumor-associated mortality. Angiogenesis in which aquaporins are involved plays a crucial role in the progression of all tumors. It is thus true that a genetic change in the AQP5 gene also determines survival, progression, metastasis and treatment response of other carcinomas. In general, any cells in the human body may become malignant and lead to a cancer. To this extent, the mechanisms and claims described here also apply to all human tumors, e.g., including the following tumors.

Tumors of the urogenital tract: examples to be mentioned here include urinary bladder carcinoma, renal cell carcinoma, prostatic carcinoma and seminoma.

Tumors of the female sex organs: breast cancer, corpus luteum carcinoma, ovarian cancer, cervical cancer.

Tumors of the gastrointestinal tract: cancer of the oral cavity, esophageal carcinoma, gastric carcinoma, hepatic carcinoma, biliary duct carcinoma, pancreatic carcinoma, colon carcinoma, rectal carcinoma.

Tumors of the respiratory tract: laryngeal carcinoma, bronchial carcinoma.

Tumors of the skin: malignant melanoma, basalioma, T-cell lymphoma.

Tumors of the hematopoietic system: Hodgkin's lymphoma and non-Hodgkin's lymphoma, acute and chronic leukemias.

Tumor diseases of the brain or nervous tissue: glioblastoma, neuroblastoma, medulloblastoma, meningeal sarcoma, astrocytoma.

Soft tissue tumors, e.g., sarcomas and tumors of the head and neck area.

### Importance of the A(−1364)C Polymorphism for Lung Diseases

As already explained above, the expression level of AQP5 is associated with great changes in the activity of the renin-angiotensinogen-aldosterone system (RAAS). Pulmonary RAAS influences pulmonary vascular tone, pulmonary capillary permeability, migration of leukocytes and fibroblast activity, so a number of diseases of the lungs such as COPD, asthma, pulmonary fibrosis, mucoviscidosis, sarcoidosis, pneumonia, ARDS, acute pulmonary edema, smoke inhalation, neurogenic pulmonary edema and other conditions can be influenced by AQP5 and genetic alterations in AQP5.

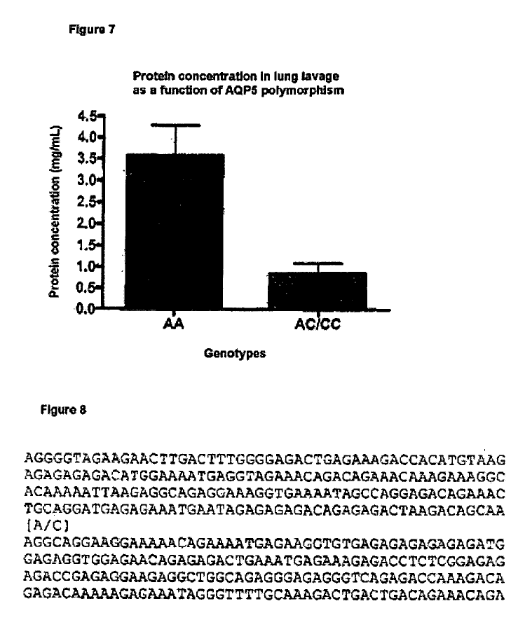

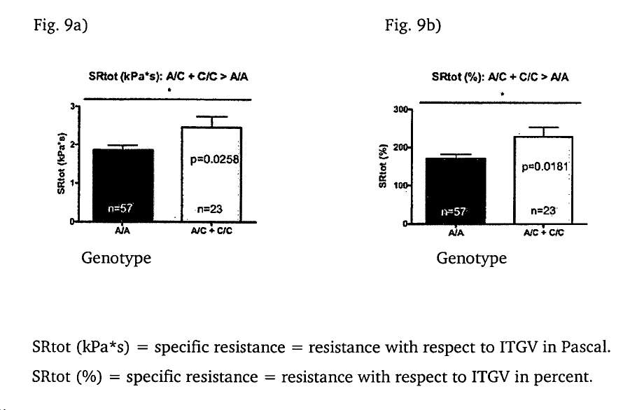

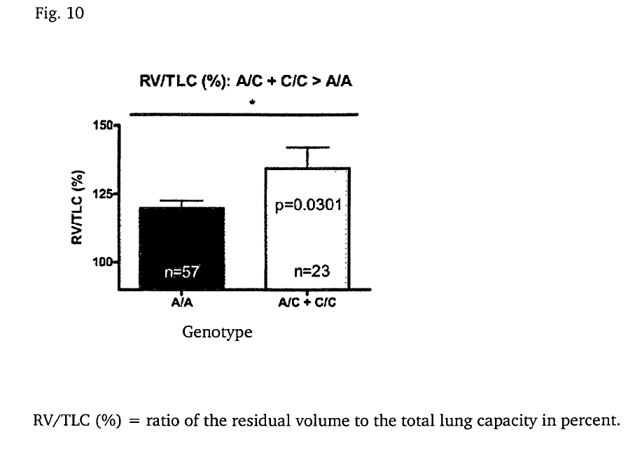

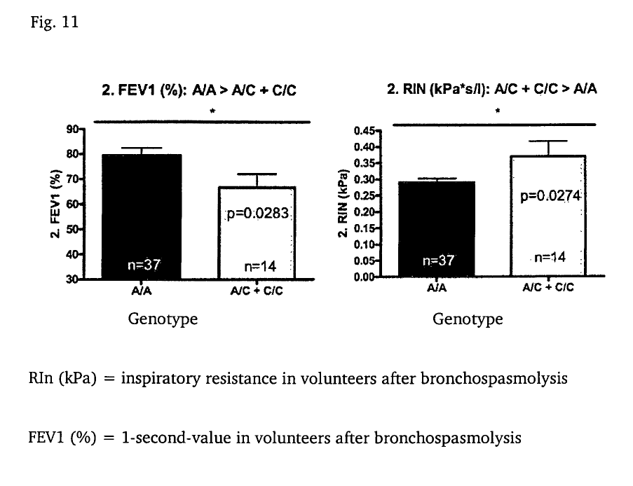

In patients with acute respiratory distress syndrome, it has been found that homozygous AA patients have a significantly higher protein content (3.57±0.8 mg/mL) in the bronchoalveolar lavage (BAL) than those with the C-allele (0.85±0.23 mg/mL; p=0.01) with acute respiratory distress syndrome (FIG. 7). The bronchoalveolar lavage (BAL) allows sampling of the surface of the terminal respiratory tract. The composition of the BAL is an indicator of the extent of the inflammatory lung damage. Important descriptive parameters include the total cell count, the differential cytology and the protein content. Although for most inflammation mediators, it has not been possible to establish a correlation with the severity of the lung damage, the protein content has proven to be a suitable parameter. Under physiological conditions, the alveolo-endothelial barrier is impermeable for neutrophilic granulocytes because of its structure. Permeability is increased during the inflammatory reaction, the alveolo-endothelial barrier is destroyed, granulocytes migrate in and increased proteins and water also enter the alveoli. The protein concentration in the lavage increases and the water content in the lungs increases. The lungs show so-called capillary leakage. The greater the protein content in the BAL, the greater is the capillary leakage and thus also the lung damage. Carriers of the C-allele thus have a survival advantage in acute respiratory distress syndrome. The cause of the increased capillary leakage is the influence of AQP5 on the pulmonary vascular tone. In ARDS patents, due to the oxygen deficiency, a pulmonary arterial hypertension (PAH) develops; this is more pronounced in patients with the AA genotype than in those with the heterozygous AC or homozygous CC genotype. In treatment of PAH, prostanoids, prostacyclines or nitrogen monoxide (NO) have been tested for their efficacy on PAH. This testing is necessary because the pulmonary vascular tone of patients responds very differently to particular drugs. AQP5-1364-SNP is responsible for some patients reacting to one medication but not to another, so the choice of medication is definitely improved. In mice deficient in AQP5, it is found that AQP5 is the protein mainly responsible for pulmonary water transport (22). AQP5 knockout mice have increased bronchoconstriction (9). The cause of this is an exacerbated mucociliary clearance because in the absence of AQP5 the viscosity of the respiratory secretion is definitely increased. Expression of AQP5 can thus strongly influence the course of conditions such as COPD, mucoviscidosis, fibrosis, asthma, pulmonary edema and pulmonary infections (pneumonia). Therapeutically, increased AQP5 expression might improve the course of the disease. On the whole, detection of genetic changes in AQP5, e.g., detection of genotypes of the A(−1364)C polymorphism, can predict the course of diseases of the lungs and the treatment suitable for the individual. It also seems possible through suitable substances to reversibly inhibit the function of aquaporin 5 (e.g., through mercury compounds). Thus excessive pulmonary water production, e.g., in smoke inhalation or neurogenic pulmonary edema can be suppressed. In a large randomized study, it has been shown that intrathoracic pulmonary water is reduced by beta-adrenergic agonists. One reason for this is the increased expression of AQP5 by beta-adrenergic agonists. Increased AQP5 expression may thus also constitute a new treatment option in acute pulmonary edema.

The composition of the bronchial secretion as well as the renin-angiotensin-aldosterone system (RAAS) have an influence on lung function, so tests were conducted to determine whether the genetic A(−1364)C polymorphism influences the pulmonary function of patients with a tentative diagnosis of obstructive pulmonary diseases.

In a prospective unicentric study, 250 volunteers underwent lung function testing by means of whole-body plethysmography as part of routine testing and also provided a mouth smear for DNA extraction and genotyping.

Of the 250 volunteers who were recruited for this study, there were 80 volunteers with a combined, i.e., central and peripheral obstruction. The lung function parameters that were determined were subsequently compared with the genotypes detected (AA, AC and CC types) in a statistical analysis).

Patients who were carriers of the C-allele with obstructive pulmonary diseases have a significantly higher resistance to overcome in respiration than do A-allele carriers and thus they have a higher degree of illness.

Furthermore, C-allele carriers had a higher ratio of the residual volume to the total lung capacity in comparison with patients who were carriers of the A-allele. This function parameter also shows a greater pathological change in lung tissue in C-allele carriers in comparison with A-allele carriers.

In addition, A-allele carriers have a better response to asthma medication in the broncholysis test than do C-allele carriers.

The oxygen content in the blood was also higher in A-allele carriers in comparison with C-allele carriers.

The A(−1364)C gene polymorphism influences lung function, the degree of illness and the response to medication in patients with lung diseases. The A(−1364)C gene polymorphism may thus be used to predict the course of lung diseases and the success of treatment.

Explanation of Abbreviations:

SRtot (kPa*s)=specific resistance=resistance with respect to the ITGV in Pascal

SRtot (%)=specific resistance=resistance with respect to the ITGV in percent

RIn (kPa)=inspiratory resistance in volunteers after bronchospasmolysis

pO2 (mmHg)=oxygen partial pressure

SaO2 (%)=oxygen saturation

FEV1 (%)=1-second value in volunteers after bronchospasmolysis

RV/TLC (%)=ratio of the residual volume to the total lung capacity in percent.

### Importance of the A(−1364)C Polymorphism for Other Diseases

The use of a genetic alteration in the human AQP5 gene may also be used for a diagnosis of the disease risk and/or the course and treatment response of a variety of other diseases. These diseases are characterized by a disturbed fluid retention across the cell membranes and/or body compartments or changes in the composition of body fluids due to altered water secretion. Disturbed fluid retention and/or shifts are observed with all forms of edema (23) (e.g., pulmonary edema, cerebral edema) but also with eye diseases (glaucoma) (24) and diseases of the inner ear with an influence on hearing ability (25). The composition of saliva is disturbed in the absence of AQP5 so that there is an increased incidence of caries and periodontosis (26).

### BIBLIOGRAPHY

1. Verkman, A S: Physiological importance of aquaporins: lessons from knockout mice. Curr Opin Nephrol Hyper-tens 9:517-522, 2000
2. Verkman, A S: Lessons on renal physiology from transgenic mice lacking aquaporin water channels. J Am Soc Nephrol 10:1126-1135, 1999
3. Verkman, A S: Role of aquaporin water channels in eye function. Exp Eye Res 76:137-143, 2003
4. Papadopoulos, M C, Krishna, S, Verkman, A S: Aquaporin water channels and brain edema. Mt Sinai J Med 69:242-248, 2002
5. Verkman, A S, Yang, B, Song, Y, Manley, G T, Ma, T: Role of water channels in fluid transport studied by phenotype analysis of aquaporin knockout mice. Exp Physiol B5 Spec No:233S-241S, 2000
6. Herrera, M, Hong, N J, Garvin, J L: Aquaporin-1 Transports NO Across Cell Membranes. Hypertension 2006
7. Liu, K, Kozono, D, Kato,', Agre, P, Hazama, A, Conversion of aquaporin 6 from an anion channel to a water-selective channel by a single amino acid substitution. Proc Nati Acad Sci USA 102:2192-2197, 2005
8. Sidhaye, V, Hoffert, J D, King, L S: cAMP has distinct acute and chronic effects on aquaporin-5 in lung epithelial cells. J Biol Chem 280:3590-3596, 2005
9. Krane, C M, Fortner, C N, Hand, A R, McGraw, D W, Lorenz, J N, Wert, S E, Towne, J E, Paul, R J, Whitsett, J A, Menon, A G: Aquaporin 5-deficient mouse lungs are hyperresponsive to cholinergic stimulation. Proc Natl Acad Sci USA 98:14114-14119, 2001
10. Moon, C, Soria, J C, Jang, S J, Lee, J, Obaidul, H M, Sibony, M, Trink, B, Chang, Y S, Sidransky, D, Mao, L: Involvement of aquaporins in colorectal carcinogenesis. Oncogene 22:6699-6703, 2003
11. Ishikawa, Y, Iida, H, Ishida, H: The muscarinic acetylcholine receptor-stimulated increase in aquaporin-5 levels in the apical plasma membrane in rat parotid acinar cells is coupled with activation of nitric oxide/cGMP signal transduction. Mol Pharmacol 61:1423-1434, 2002
12. Marrades, M P, Milagro, F I, Martinez, J A, Moreno-Aliaga, M J: Differential expression of aquaporin 7 in adipose tissue of lean and obese high fat consumers. Biochem Biophys Res Commun 339:785-789, 2006
13. MacDougald, O A, Burant, C F: Obesity and metabolic perturbations after loss of aquaporin 7, the adipose glycerol transporter. Proc Natl Acad Sci USA 102:10759-10760, 2005
14. Hibuse, T, Maeda, N, Funahashi, T, Yamamoto, K, Nagasawa, A, Mizunoya, W, Kishida, K, Inoue, K, Kuriyama, H, Nakamura, T, Fushiki, T, Kihara, S, Shimomura, I: Aquaporin 7 deficiency is associated with development of obesity through activation of adipose glycerol kinase. Proc Nati Acad Sci USA 102:10993-10998, 2005
15. Hara, M, Chikuma, M, Verkman, A S: Aquaporin-3 functions as a glycerol transporter in mammalian skin. Biol Cell 97:479-486, 2005
16. Hara, M, Verkman, A S: Glycerol replacement corrects defective skin hydration, elasticity, and barrier function in aquaporin-3-deficient mice. Proc Natl Acad Sci USA 100:7360-7365, 2003
17. Hara, M, Ma, T, Verkman, A S: Selectively reduced glycerol in skin of aquaporin-3-deficient mice may account for impaired skin hydration, elasticity, and barrier recovery. J Biol Chem 277:46616-46621, 2002

18. Hoque, M O, Soria, J C, Woo, J, Lee, T, Lee, J, Jang, S J, Upadhyay, S, Trink, B, Monitto, C, Desmaze, C, Mao, L, Sidransky, D, Moon, C: Aquaporin 1 is overexpressed in lung cancer and stimulates NIH-3T3 cell proliferation and anchorage-independent growth. Am J Pathol 168:1345-1353, 2006
19. Mazal, P R, Susani, M, Wrba, F, Haitel, A: Diagnostic significance of aquaporin-1 in liver tumors. Hum Pathol 36:1226-1231, 2005
20. Yang, J H, Shi, Y F, Chen, X D, Qi, W J: The influence of aquaporin-1 and microvessel density on ovarian carcinogenesis and ascites formation. Int J Gynecol Cancer 16 Suppl 1:400-405, 2006
21. Clapp, C, Martinez de la, E G: Aguaporin-1: a novel promoter of tumor angiogenesis. Trends Endocrinol Metab 17:1-2, 2006
22. Ma, T, Fukuda, N, Song, Y, Matthay, M A, Verkman, A S: Lung fluid transport in aquaporin-5 knockout mice. J Clin Invest 105:93-100, 2000
23. Lo, A C, Chen, A Y, Hung, V K, Yaw, L P, Fung, M X, Ho, M C, Tsang, M C, Chung, S S, Chung, S K: Endothelin-1 overexpression leads to further water accumulation and brain edema after middle cerebral artery occlusion via aquaporin 4 expression in astrocytic end-feet. J Cereb Blood Flow Metab 25:998-1011, 2005
24. Stamer, W D, Snyder, R W, Smith, B L, Agre, P, Regan, J W: Localization of aquaporin CHIP in the human eye: implications in the pathogenesis of glaucoma and other disorders of ocular fluid balance. Invest Ophthalmol Vis Sci 35:3867-3872, 1994
25. Mhatre, A N, Stern, R E, Li, J, Lalwani, A K: Aquaporin 4 expression in the mammalian inner ear and its role in hearing. Biochem Biophys Res Commun 297:987-996, 2002
26. Culp, D J, Quivey, R Q, Bowen, W H, Fallon, M A, Pearson, S K, Faustoferri, R: A mouse caries model and evaluation of aqp5−/− knockout mice. Caries Res 39:448-454, 2005

1. An in-vitro method for predicting disease risks, disease courses and the response of an individual patient to pharmacological and nonpharmacological treatments, said method comprising screening for a polymorphism in the AQP5 gene on the human chromosome 12q13.

2. The method according to claim 1, wherein screening for a polymorphism is performed in the promoter region of the human AQP5 gene.

3. The method according to claim 2, wherein screening is performed for A(−1364)C polymorphism in the promoter region of the human AQP5 gene.

4. The method according to claim 3, wherein the disease risk and disease courses are selected from diseases and disorders of the cardiovascular system, tumor diseases, pulmonary diseases and disturbances in the water and electrolyte equilibrium in the human body.

5-8. (canceled)
